SEQUENCE LISTING (20 sequences)

SEQ ID NO: 1 - DNA, 25 bp, Artificial Sequence
  Primer ACTBF
tggtgatggaggaggtttagtaagt

SEQ ID NO: 2 - DNA, 23 bp, Artificial Sequence
  Primer ACTBR
aaccaataaaacctactcctccc

SEQ ID NO: 3 - DNA, 34 bp, Artificial Sequence
  Primer INSMETHF
cggaaattgtagttttagtttttagttatttgtc

SEQ ID NO: 4 - DNA, 30 bp, Artificial Sequence
  Primer INSMETHR
cctaaaaaactaaaaactactaaacccccg

SEQ ID NO: 5 - DNA, 34 bp, Artificial Sequence
  Primer INSUNMETHF
tggaaattgtagttttagtttttagttatttgtt

SEQ ID NO: 6 - DNA, 30 bp, Artificial Sequence
  Primer INSUNMETHR
cctaaaaaactaaaaactactaaaccccca

SEQ ID NO: 7 - DNA, 25 bp, Artificial Sequence
  Primer IAPPF
tgttattagttattaggtggaaaag

SEQ ID NO: 8 - DNA, 23 bp, Artificial Sequence
  Primer IAPPMETHR
taaaaaatttaccaaacgctacg

SEQ ID NO: 9 - DNA, 23 bp, Artificial Sequence
  Primer IAPPUNMETHR
taaaaaatttaccaaacactaca

SEQ ID NO: 10 - DNA, 20 bp, Artificial Sequence
  Primer GCKMETHF
aatgtcgagcggcgtttgag

SEQ ID NO: 11 - DNA, 25 bp, Artificial Sequence
  Primer GCKUNMETHF
aggaaatgttgagtggtgtttgagt

SEQ ID NO: 12 - DNA, 30 bp, Artificial Sequence
  Primer GCKR
atcctctcccttctataacctaaaaacaac

SEQ ID NO: 13 - DNA, 18 bp, Artificial Sequence
  Primer KCNJ11METHF
cgggtttcggtttcgttc

SEQ ID NO: 14 - DNA, 20 bp, Artificial Sequence
  Primer KCNJ11METHR
cacgaacgaacaaacaaacg

SEQ ID NO: 15 - DNA, 23 bp, Artificial Sequence
  Primer KCNJ11UNMETHF
tgggttttggttttgtttgttgt

SEQ ID NO: 16 - DNA, 23 bp, Artificial Sequence
  Primer KCNJ11UNMETHR
acccacaaacaaacaaacaaaca

SEQ ID NO: 17 - DNA, 21 bp, Artificial Sequence
  Primer ABCC8METHF
taggaagacgtgcggtattac

SEQ ID NO: 18 - DNA, 20 bp, Artificial Sequence
  Primer ABCC8METHR
ctacgacaacgaaaaccact

SEQ ID NO: 19 - DNA, 24 bp, Artificial Sequence
  Primer ABCC8UNMETHF
gagtaggaagatgtgtggtattat

SEQ ID NO: 20 - DNA, 23 bp, Artificial Sequence
  Primer ABCC8UNMETHR
cttctacaacaacaaaaaccact

(19) United States
(12) Patent Application Publication
(10) Pub. No.: US 2024/0170100 A1
(43) Pub. Date: May 23, 2024

(54) METHOD FOR MONITORING PANCREATIC BETA-CELL DESTRUCTION IN DISEASE PREDICTION/DIAGNOSIS/PROGNOSIS OF TYPE 2 DIABETES MELLITUS

(71) Applicants: Ekaterini Chatzaki, Alexandroupolis (GR); Democritus University of Thrace, Alexandroupolis (GR)

(72) Inventors: Ekaterini Chatzaki, Alexandroupolis (GR); Makrina Karaglani, Alexandroupolis (GR); loannis Tsamardinos, Crete (GR)

(21) Appl. No.: 18/563,294

(22) PCT Filed: May 20, 2022
(86) PCT No.: PCT/EP22/00048

§ 371 Date: Nov. 21, 2023

(30) Foreign Application Priority Data
May 24, 2021 (EP) 21386030.7

(51) Int. Cl. G16B 40/20; G16H 50/20

(52) U.S. Cl. CPC G16B 40/20; G16H 50/20

(57) ABSTRACT

The patent relates to a computer implemented method for early diagnosis of diabetes mellitus type 2 (T2DM) and β-pancreatic cell loss monitoring. The method employs Machine Learning tools to identify specific biomarkers related to circulating cell-free DNA (ccfDNA) to build accurate diagnostic/monitoring predictive biosignatures/models for clinical application. The models are further enriched with demographical/lifestyle/pathological/clinical subject data to achieve higher discriminating capacity. AutoML analysis of data delivered a five-feature biosignature, including GCK. IAPP and KCNJ11 gene methylation identified in ccfDNA and age and BMI, accurately discriminating T2DM patients from healthy individuals. This leads to a methylation-specific PCR based methodology for diagnosis/prognosis/monitoring of T2DM using liquid biopsy biomaterial.

### FIELD OF THE INVENTION

The present invention relates to an innovative method for type 2 Diabetes Mellitus (T2DM) prediction/diagnosis/prognosis based on liquid biopsies and a specific automated computer implemented predictive model. In particular, the present invention relates to a novel method based on a machine learning-built model, integrating diabetes-related/pancreatic beta-cell (β-cell) gene methylation detected in circulating cell-free DNA (ccfDNA) from liquid biopsies in combination to selected lifestyle/clinical/demographical parameters, showing high sensitivity, specificity and accuracy in discriminating T2DM patients from healthy subjects. The method is capable of β-cell destruction detection and monitoring and can be used for early T2DM diagnosis, disease prediction and prognosis.

### BACKGROUND OF THE INVENTION

T2DM is expected to be one of the leading causes of death globally by 2030, while it has been recognized globally as a serious pathological entity with 425 million adults having diabetes while the half remain undiagnosed(1). Therefore it is of crucial importance the early diagnosis of T2DM for provision of early and appropriate medical treatment to the suffering patients.

T2DM is characterized by inadequate B-cell function, insulin insensitivity and chronic inflammation, all of which progressively lead to impaired glucose homeostasis(2). In post-mortem specimens from patients with T2DM, β-cell mass is reduced by 30-40% compared with specimens from non-diabetic subjects(3). It is found that increased β-cell apoptosis and reduced functional capacity of the remaining cells are important factors that indicate progression of the disease(4). However, the detection of the loss of β-cell mass is currently possible after the development of hyperglycemia which means significant β-cell mass and therefore significant development of the disease limiting the possibility of early intervention.

Established biomarkers for monitoring T2DM progression like HbAlc levels are unable to detect ongoing β-cell destruction and its dynamics in islets. Thus, as the number of people with T2DM worldwide keeps on rising, there is an emerging need for additional more precise monitoring of β-cell death for disease prediction, early diagnosis and leading therapeutic decisions.

The disclosed method is using the methylation profile of diabetes-related genes, assessed via methylation specific Polymerase Chain Reaction (MSP) or sequencing or other method, to build a highly performing tool/model/classifier/biosignature of clinical value on diabetes. The use of liquid biopsies (ccfDNA) rather than peripheral blood leukocytes is of particular importance, as methylation is a tissue specific-event and its detection in liquid biopsy biomaterial differs dramatically that its detection in genomic DNA from other sources. Gene methylation status detected in ccfDNA can reflect methylation status in the tissue of origin, in this case the beta-pancreatic islets. Further, integrating those ccfDNA gene methylation patterns with lifestyle/clinical/demographical data to be analysed by automated machine learning, allowed the formation of predictive models with the highest reported sensitivity and specificity in recognizing T2DM patients from healthy subjects.

### PRIOR ART

Some attempts have been previously made for early diagnosis of Diabetes and monitoring using minimally invasive biomarkers. Towards this direction, epigenetic information detected in ccfDNA is currently tested as a clinically valuable biomarker of pancreatic β-cell death in type 1 Diabetes Mellitus (T1DM) by several groups(5-12). These biomarkers rely on the principles that a. cells release amounts of their DNA into the bloodstream and/or other biofluids where they are easily accessible(13) and b. each cell type has a unique methylome that affects gene expression(14). Akirav's group (33), has identified a unique DNA CpG methylation pattern as compared to other cells in the blood upon pancreatic β-cell death. The method used comprises extracting and purifying DNA; treating the extracted purified DNA with bisulfite to convert cytosine to uracil; amplifying a region of INS gene on the bisulfite-treated DNA by PCR; determining a quantitative relationship between the DNA portion having the unique DNA CpG methylation pattern to the DNA portion lacking the unique DNA CpG methylation pattern, by employing DNA CpG methylation pattern-specific probes; and computing a difference between the DNA portion having the unique DNA CpG methylation pattern and the DNA portion lacking the unique DNA CpG methylation pattern. A general method of detecting death of a cell type or tissue of any type in a subject is disclosed from Dor (34), wherein a methylome atlas comprises methylation data from several cell types including pancreatic β-cells. The existing methods are based on the selection of a single methylation biomarker and this results to high uncertainty in a disease diagnosis including Diabetes.

Machine learning (ML) is the application of artificial intelligence on data analysis to build trained models (15). ML has penetrated biomarker discovery in many diseases(16-18) and in diabetes(19, 20). ML had been used for building models useful for the prediction and diagnosis of diseases such as Alzheimer's disease(21), lung and breast cancer(22, 23). ML has been used previously (35) to form a risk predictive model of T2DM based on data from insurance claim databases. However, the model does not disclose the specific data used or a specific model but it is rather used as a general framework analyzing any of the given data. The method needs many data and the accuracy of disease detection is low as compared to the biomarker-based methods.

Tsamardinos et al., has developed an automated Machine Learning tool, JADBio which utilizes a number of different algorithms to find the best matching ML algorithm to classify the data. It is automated which means that takes a new case and based on a training dataset, classifies it into one category. However in his studies, no reference to diabetes Mellitus, targeted gene methylation or liquid biopsies is made since it is in a different field.

A study of Lai et al. presents predictive models for diabetes mellitus using machine learning techniques. Using the most recent records of 13,309 Canadian patients aged between 18 and 90 years, along with their laboratory information (age, sex, fasting blood glucose, body mass index, high-density lipoprotein, triglycerides, blood pressure, and low-density lipoprotein), the group built predictive models using Logistic Regression and Gradient Boosting. The dataset analysed is of different modalities than in the disclosed invention (no methylation (epigenetic) measurements) in different biomaterial (no liquid biopsy, ie cell-free DNA). The analysis does not involve automated machine learning techniques.

In a review paper of Ahmed et al 2020 they mention the detection of Diabetes by separate GCK, INS and KCNJ11methylation data, drawn by studies in peripheral blood leukocytes but not in liquid biopsy cell-free DNA. Methylation is a tissue specific-event and its detection in liquid biopsy material (ccfDNA) differs dramatically that its detection in genomic DNA from other sources such as blood cells. Gene methylation status detected in ccfDNA can reflect methylation status in the tissue of origin, in this case the beta-pancreatic islets. The review paper mentions also the expression regulation of LAPP by miRNAs but they do not refer to any data on the methylation status of IAPP irrelevant to its use as an epigenetic biomarker.

Furthermore, ABCC8 (ATP Binding Cassette Subfamily C Member 8) is not referred to at all. Most importantly, described findings do not address the diabetes classification through a panel of biomarkers, or their combination with clinicopathological or demographic/lifestyle parameters, as here.

Harleen Kaur and Vinita Kumarihttps [38] performed predictive modelling and analytics for diabetes using a machine learning approach, with the aim to develop trends and recognise patterns of risk factors, rather than detect the actual onset of diabetic early phenotype, ie initiation of beta-cell destruction. They used some supervised Machine Learning algorithms, rather than automated machine learning (which employs all possible algorithms). The dataset analysed was extracted from medical records including eight different risk factors: number of times pregnant, plasma glucose concentration of two hours in an oralglucose tolerance test, diastolic blood pressure, triceps skin fold thickness, two-hour seruminsulin, body mass index, diabetes pedigree function and age. Neither gene methylation (epigenetic biomarkers) data, nor cell-free DNA (liquid biopsy) measurements were used.

Finally, Syed et al., reported that circulating unmethylated CHTOP and INS DNA fragments provide evidence of possible islet cell death in youth with obesity and diabetes. They have studied the methylation of two genes (rather than a panel of five as disclosed here with technical effect of higher accuracy). The Naïve Bayes classifier (machine learning algorithm), mentioned in the article was not used on ccfDNA methylation data (liquid biopsy) as here, but rather on pancreatic cell methylation data. They have studied mainly type 1 Diabetes Mellitus (T1DM) subjects (not type 2 Diabetes Mellitus which is a different disease than type 1). Only one gene, namely INS methylation, is shown to differ in liquid biopsies (ccfDNA) between lean and obese or T2DM subjects. This gene was not included in the predictive model in the disclosed method. Most importantly, as Syed's study focuses on T1DM, the study group includes exclusively children and young individuals (Age7-<20 years old) rather than adults as in the disclosed invention (T2DM study group's age 64±8 years).

The novelty of the disclosed invention includes:
the use of a panel of (epigenetic) methylation markers (two of which never studied before),
their use in combination to demographic and lifestyle parameters never attempted before,
the use of liquid biopsies (ccfDNA) rather than peripheral blood leukocytes to study gene methylation biomarkers,
the exclusive focus on adult patients and type 2 diabetes for liquid biopsies,
the use of machine learning classification analysis on T2DM liquid biopsy datasets,
the use of automated machine learning, never used before in type 2 diabetes mellitus datasets.

### SUMMARY OF THE INVENTION

The present invention was made in view of the prior art described above and the object of the present invention is to provide a method of improved performance over the prior art in detecting efficiently pancreatic β-cell damage and as such capable of early diagnosis (before the onset of clinical symptoms), prognosis, prediction or monitoring of T2DM, reliably and with higher sensitivity/specificity/accuracy that existing methods.

To solve the problem, we focus on liquid biopsy experimental parameters related to gene methylation detected in ccfDNA isolated from the blood of T2DM patients and healthy subjects. The levels of ccfDNA were directly quantified in serum by fluorometry. Methylation of five diabetes-related β-pancreatic cell genes used as biomarkers, namely INS (insulin), IAPP (Islet Amyloid Polypeptide-Amylin), GCK (Glucokinase), KCNJ11 (Potassium Inwardly Rectifying Channel Subfamily J Member 11) and ABCC8 (ATP Binding Cassette Subfamily C Member 8) were detected via quantitative SYBR Green-based methylation specific PCR. Then, ad hoc Automated ML (AutoML) technology (JADBio), which automatically applies a number of known algorithms to deliver a model, was applied on the ccfDNA experimental parameters to build accurate diagnostic/monitoring predictive models/biosignatures/classifiers of clinical value on diabetes. An exceptionally high classifying performance/predicting capacity as compared to the prior art, was achieved when specific 2 or 3 of the above biomarkers were combined. In specific, it was found that the inclusion of GCK methylation and one or both from LAPP and KCNJ11 methylation provided significantly higher discrimination performance between T2DM patients and healthy subjects [three feature model's AUC 0.892 (95% CI 0.848-0.934)] as compared to GCK methylation alone [AUC 0.849 (95% CI 0.803-0.892)] or combined with other biomarkers. In a further ML analysis, we combined these ccfDNA experimental biomarkers with lifestyle/clinical/demographical data (i.e., age, gender, Body Mass Index—BMI, smoking etc.) and the predictive capacity of the resulting models was further improved. The best predictive model found was based on a best performing five-feature biosignature selected via Classification Random Forests algorithm, including GCK, IAPP and

KCNJ11 methylation and age and BMI, showing high performance in discriminating between T2DM patients and healthy subjects with an AUC of 0.927 (95% CI 0.874-0.967) and an average Precision of 0.951 (95% CI 0.914-0.980). A best interpretable model of five features was also built via Ridge Logistic Regression algorithm reaching an AUC of 0.915 (95% CI 0.868-0.957) and an average Precision of 0.941 (95% CI 0.901-0.975). This biosignature's features included GCK, IAPP and KCNJ11 methylation, smoking status and BMI.

Comparison of the results to existing studies showed that models built here, including GCK methylation combined with other parameters from the above, are superior in T2DM prediction performance than any other reported so far in the prior art.

### BRIEF DESCRIPTION OF DRAWINGS

FIG. 1 Workflow of the disclosed study.

FIG. 2 A. Box plot of serum ccfDNA levels between healthy subjects and T2DM patients (p=0.552). B. Box plot of INS methylation between healthy subjects and T2DM patients (p=0.001). C. Box plot of LAPP methylation between healthy subjects and T2DM patients (p<0.001). D. Box plot of GCK methylation between healthy subjects and T2DM patients (p<0.001). E. Box plot of KCNJ11 methylation between healthy subjects and T2DM patients (p=0.001). F. Box plot of ABCC8 methylation between healthy subjects and T2DM patients (p=0.534).

FIG. 3 A. ROC curve of ccfDNA levels reaching an AUC of 0.527 (95% CI 0.438-0.616). B. ROC curve of INS gene reaching an AUC of 0.650 (95% CI 0.562-0.737). C. ROC curve of LAPP gene reaching an AUC of 0.727 (95% CI 0.649-0.805). D. ROC curve of GCK gene reaching an AUC of 0.848 (95% CI 0.787-0.910). E. ROC curve of KCNJ11 gene reaching an AUC of 0.712 (95% CI 0.619-0.806). F. ROC curve of ABCC8 gene reaching an AUC of 0.528 (95% CI 0.439-0.617).

FIG. 4 Predictive modelling results of the three-feature (GCK, IAPP and KCNJ11 methylation) best interpretable model A. ROC curve reaching an AUC of 0.892 (95% CI 0.848-0.934), B. Principal Component Analysis (PCA) plot depicting discrimination between T2DM patients and healthy subjects, C. Feature Importance plot of the features of the model. Feature importance is defined as the percentage drop in predictive performance when the feature is removed from the model, D. Probabilities box plot of out-of-sample predictions.

FIG. 5 Predictive modelling results of the five-feature (GCK, LAPP and KCNJ11 methylation and age and BMI) best performing model A. ROC curve reaching an AUC of 0.927 (95% CI 0.874-0.967), B. PCA plot depicting discrimination between T2DM patients and healthy subjects, C. Feature Importance plot of the features of the model. Feature importance is defined as the percentage drop in predictive performance when the feature is removed from the model, D. Probabilities box plot of out-of-sample predictions.

FIG. 6. Sequence listing

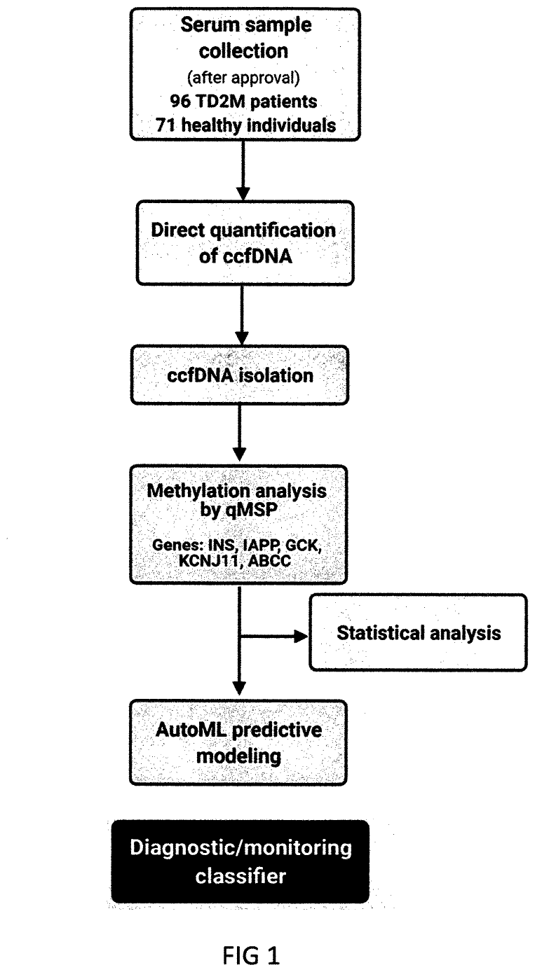

### DETAILED DESCRIPTION

The development of the method was based on a cohort of adult T2DM patients which was compared to a group of age- and sex matched healthy subjects.

In one embodiment, a computer implemented method suitable for monitoring pancreatic β-cell destruction of value in disease prediction, prognosis and early diagnosis of T2DM, was developed, said method comprising:
training a machine learning tool to minimize false positives and to maximize true positives using input parameters and a training dataset, the machine learning tool running on a computer system comprising a CPU processor and storage memory,
said training dataset including data from patients diagnosed with T2DM and healthy subjects,
using said machine learning tool to create a prediction model that predicts, given at least two or more input parameters, pancreatic beta-cell destruction indicative of the onset or progression of T2DM,
storing the prediction model in at least one non-transitory processor-readable storage medium on a computer system or on the cloud,
and classifying a new case as positive or negative concerning beta-pancreatic cell destruction, based on the said prediction model,
wherein the input parameters comprising at least of two gene methylation biomarkers (biomarker based on the methylation status of a gene) as detected in circulating cell free DNA.
A new case are the data of is a case of a gene methylation biomarkers (biomarker based on the methylation status of a gene) of a new sample as detected in circulating cell free DNA to be determined if it is positive or negative concerning beta-pancreatic cell destruction.

In a further embodiment the above Computer Implemented method for monitoring pancreatic β-cell destruction is used and said biomarkers are obtained by a method comprising:
Providing a biological fluid sample from a subject suspected of having or developing or diagnosed with said disease or a subject known not to have the said disease
Directly quantifying circulating cell free DNA (ccfDNA)
Extracting ccfDNA
Treating the said DNA to convert demethylated cytosines to uracils while sparing the methylated cytosines
Performing a methylation specific detection to measure gene methylation
Incorporate the said gene methylation measurements of to the trained machine learning tool as the input parameters.

In further embodiments, different Machine learning tools (machine learning classification algorithms) are used to build the predictive models. Any machine learning classification classification algorithm can be used and in different embodiments the following: Decision Tree, k-Nearest Neighbors (k-NN), Gradient Boosting Machine (GBM), linear kernel Support Vector Machine (SVM-linear), Radial Basis Function (RBF) kernel Support Vector Machine, Artificial Neural Network (ANN), Multifactor Dimensionality Reduction (MDR), naive Bayes, Classification And Regression Tree (CART) and preferably Support Vector Machine (SVM) or (Classification) Random Forest (RF) or Logistic Regression (LR).

In one embodiment the biological sample used to obtain some of the biomarkers in the above methods comprises a body fluid and in a further embodiment blood sample and in further embodiments serum or plasma and combinations thereof.

In further embodiments, in the above computer implemented methods the biomarkers used to train the Machine learning tools are diabetes-related genes, in further embodiments are pancreatic β-cell genes and in further embodiments the genes GSK and/or LAPP and/or KCNJ11 and combinations thereof.

The above prediction model has an AUC (Area under Curve) of 0.884 or higher indicating the high predictive capability of the model.

In further embodiments the said methylation measurements of the genes are expressed either qualitatively or quantitatively as indexes of methylation levels and in further embodiments by any of the following quantification methods/formulas and combinations thereof:
The relative quantity (RQsample) of demethylated alleles using the 2^{-ΔΔCT} method where the ΔΔCT values were generated for each target after normalization by reference gene values.
The demethylation index was calculated using the formula: 2^{(methylated Ct-unmethylated Ct)}.
The percentage of methylation in a sample using the formula:

$$1 - \frac{1}{1 + {2}^{( - Δ Ct )}} \times 100 \% ,$$

where ΔCt=Ct_{unmethylated}−Ct_{methylated}

In further embodiments the reference gene is any housekeeping gene and in further embodiments the reference genes used are ACTB or GADPH or COL2A1 gene respectively. In one embodiment the methylation specific detection to measure gene methylation and its levels is conducted by sequencing and in a further embodiment by PCR-based technology.

In further embodiments the input parameters further comprising lifestyle and/or personal and/or demographic and/or clinical and/or clinicopathological data of the subjects and in additional embodiments, the BMI and/or the smoking status and/or the age of the subjects and combinations thereof.

In a further embodiment a method is used to test the biomarkers of a subject on the previous trained predictive model to early diagnose/predict the development of type 2 diabetes mellitus on the said subject. The method comprises of the steps
Providing a biological fluid sample already obtained from a subject suspected of having or having said disease
Directly quantifying circulating cell free DNA (ccfDNA)
Extracting ccfDNA
Treating the said DNA to convert demethylated cytosines to uracils while sparing the methylated cytosines
Performing a methylation specific detection based on PCR or sequencing-based technology and in further embodiments quantitative methylation specific PCR, to measure the methylation of genes and in further embodiments pancreatic β-cell genes and in further embodiments of the genes GSK and/or IAPP and/or KCNJ11 and combinations thereof
Incorporate input data comprising of measurements of the status or the amount of methylation of at least two of the said selected genes as data input parameters into the trained predictive model built by the above predictive machine learning tool
Produce a score using the trained machine learning tool of the previous embodiments, indicative for clinical prognosis or diagnosis.

In this embodiments the data used are in accordance to the data input required in the predictive model.

In a further embodiment the biological sample used to obtain some of the biomarkers of the subject to be tested on the trained predictive model comprises a body fluid and in a further embodiment blood sample and in further embodiments serum or plasma and combinations thereof

In a further embodiment the input data of the biomarkers of the subject to be tested on the trained predictive model comprises further of the lifestyle and/or personal and/or demographic data and/or clinical and/or clinicopathological data of the subject and in further embodiments the BMI and/or the smoking status and/or the age of the subjects and combinations thereof and in accordance to the predictive model.

In further embodiments the input data of the biomarkers of the subject to be tested on the trained predictive model comprises of the said methylation measurements of the genes, expressed either qualitatively or quantitatively as indexes of methylation levels and in further embodiments by any of the following quantification methods/formulas and combinations thereof and in accordance to the methods/formulas used for the training of the predictive model.
The relative quantity (RQsample) of demethylated alleles using the 2−ΔΔCT method where the ΔΔCT values were generated for each target after normalization by reference gene values.
The demethylation index was calculated using the formula: 2(methylated Ct−unmethylated Ct).
The percentage of methylation in a sample using the formula: 1−1/(1+2\hat{ }((−ΔCt))×100%, where ΔCt=Ct_{unmethylated}−Ct_{methylated}

In a further embodiment a computer implemented method was used comprising of the input of the biomarkers of the tested for diabetes T2DM having or going to develop subject in the trained predictive model as manual input dataset or as data stored in a database or a non-transient computer memory in order the predictive model to produce a score using the trained machine learning tool of the previous embodiments, indicative for clinical prognosis or diagnosis.

In a specific embodiment, the study's groups consisted of 96 T2DM patients on treatment and 71 healthy subjects of similar age without history of diabetes. All samples were of Caucasian origin. T2DM was diagnosed according to the American Diabetes Association (ADA) guidelines(24). Lifestyle/personal/clinical/demographic data of study groups are shown in Table 1.

TABLE 1
Lifestyle/personal/clinical/demographic data of study groups.

| | T2DM patients | Healthy subjects |
|---|---|---|
| | (n = 96) | (n = 71) |
| Gender (male) | 58 (60) | 50 (70) |
| Age (years) | 64 ± 8 | 62 ± 10 |
| BMI (kg/m²) | 30.9 ± 4.7 | 28.4 ± 5.2 |
| Smoking | 16 (17) | 25 (39) |
| Glucose (mg/dL) | 138 ± 37 | 85 ± 13 |
| C-peptide (ng/mL) | 4.1 ± 3.2 | — |
| HbA1c (%) | 7.2 ± 1.1 | — |
| Diabetes duration (years) | 14 ± 9 | — |
| Diabetes complication | 25 (26) | — |
| (presence) | | |
| Diabetes therapy (yes) | 96 (100) | — |
| Oral therapy (metformin, | 96 (100) | — |
| etc.) | | |
| Insulin | 42 (44) | — |

Inclusion criteria of the study included age more than 25 and up to 75 years old and ability to give informed consent. Exclusion criteria of all participants included the presence of a (another) chronic disease, underlying malignancies and systemic lupus erythematosus.

ccfDNA was isolated and measured in body fluids. In a specific embodiment, serum samples were obtained within 2 hours of blood sampling through centrifugation at 3,000×g for 10 min. An additional high-speed centrifugation step at 14,000×g for 10 min was performed to remove any cellular debris and contaminants, like gDNA from damaged blood cells. Serum samples were stored at −80ºC until further use. Following, ccfDNA was quantified. Several direct or indirect methods can be used for quantifying ccfDNA in body fluids with comparable results.

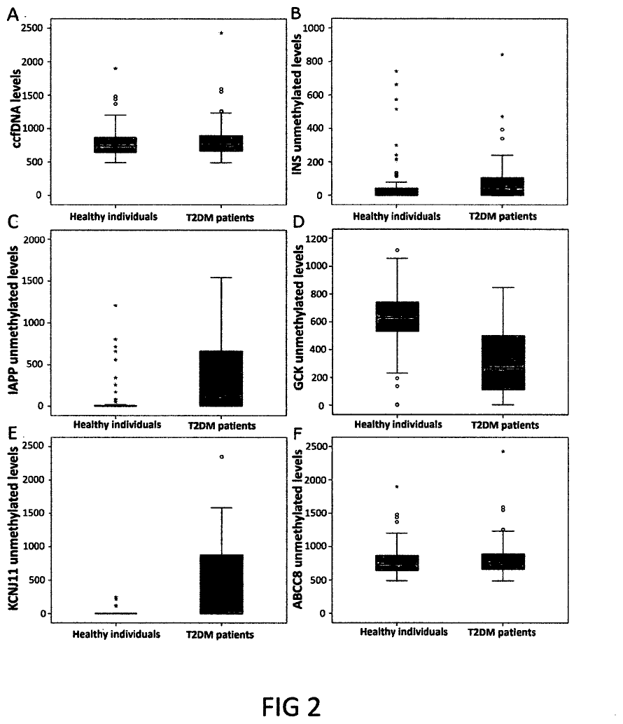

In a specific embodiment, ccfDNA was directly quantified in 20 μL of serum, utilizing the Quant-iT™ dsDNA High-Sensitivity Assay Kit in Qubit® 3.0 Fluorometer (Invitrogen, Karlsruhe, Germany). A standard curve was generated using kit provided standards (Ong/ul and 10 ng/μl). Experimental data from measured ccfDNA levels are shown in FIG. 2A. ccfDNA levels did not differ between healthy subjects and diabetes patients [700(400-1590) ng/ml vs. 820(490-2430) ng/ml, respectively, p=0.552]

Following, ccfDNA was extracted from body fluids. Several methods can be used. In a specific embodiment, ccfDNA was extracted from 600 μl of serum using the QIAamp® Blood Mini kit (Qiagen, Hilden, Germany) in a final elution volume of 30 μl. The extracted ccfDNA was stored at −20° C. until further use.

TABLE 2
Primer sequences, genomic locations, and related references (where relevant) used for PCR
assays.

| | Primer sequence | Genomic Location | |
|---|---|---|---|
| Primer name | (5′-3′) | (GRCh38) | Ref. |
| ACTBF | TGG TGA TGG AGG AGG TTT AGT AAG T | 7:5,558,705:−1 | (25) |
| ACTBR | AAC CAA TAA AAC CTA CTC CTC CC | 7:5,558,838:−1 | |
| INSMETHF | CGG AAA TTG TAG TTT TAG TTT TTA GTT ATT | 11:2,159,776:−1 | (8) |
| | TGT C | | |
| INSMETHR | CCT AAA AAA CTA AAA ACT ACT AAA CCC | 11:2,159,922:−1 | |
| | CCG | | |
| INSUNMETHF | TGG AAA TTG TAG TTT TAG TTT TTA GTT ATT | 11:2,159,776:−1 | |
| | TGT T | | |
| INSUNMETHR | CCT AAA AAA CTA AAA ACT ACT AAA CCC | 11:2,159,922:−1 | |
| | CCA | | |
| IAPPF | TGT TAT TAG TTA TTA GGT GGA AAA G | 12:21,378,228:1 | |
| IAPPMETHR | TAA AAA ATT TAC CAA ACG CTA CG | 12:21,378,304:1 | |
| IAPPUNMETHR | TAA AAA ATT TAC CAA ACA CTA CA | 12:21,378,304:1 | |
| GCKMETHF | AAT GTC GAG CGG CGT TTG AG | 7:44,153,026:−1 | (9) |
| GCKUNMETHF | AGG AAA TGT TGA GTG GTG TTT GAG T | 7:44,153,022:−1 | |
| GCKR | ATC CTC TCC CTT CTA TAA CCT AAA AAC AAC | 7:44,153,136:−1 | |
| KCNJ11METHF | CGG GTT TCG GTT TCG TTC | 11:17,389,140:1 | |
| KCNJ11METHR | CAC GAA CGA ACA AAC AAA CG | 11:17,389,253:1 | |
| KCNJ11UNMETHF | TGG GTT TTG GTT TTG TTT GTT GT | 11:17,389,140:1 | |
| KCNJ11UNMETHR | ACC CAC AAA CAA ACA AAC AAA CA | 11:17,389,254:1 | |
| ABCC8METHF | TAG GAA G?C GTG CGG TAT TAC | 11:17,476,660:1 | |
| ABCC8METHR | CTA CGA CAA CGA AAA CCA CT | 11:17,476,743:1 | |
| ABCC8UNMETHF | GAG TAG GAA GAT GTG TGG TAT TAT | 11:17,476,657:1 | |
| ABCCSUNMETHR | CTT CTA CAA CAA CAA AAA CCA CT | 11:17,476,743:1 | |

F forward, R reverse, METH methylated, UNMETH unmethylated

Following, gene methylation was detected in ccfDNA after converting by sodium bisulfite. In one embodiment, 20 μl of extracted ccfDNA were treated with sodium bisulfite (SB) using the EZ DNA Methylation-Gold™ kit (ZYMO Research Co., CA, USA) in a final elution volume of 10 μl. In each reaction, CpGenome™ Human methylated and non-methylated DNA controls (Merck Millipore, Darmstadt, Germany) were included as negative and positive control samples, respectively. The SB-treated ccfDNA was stored at −80° C. until further use.

Different PCR-based (i.e. methylation-specific PCR MSP, Methylight and others) or sequencing-based (i.e. bisulfite sequencing, pyrosequencing and others) methodologies are available for detecting gene methylation on DNA. In one embodiment, a methylation-independent PCR assay for the housekeeping gene ß-actin (ACTB) was used in order to verify quality and quantity of SB-treated ccfDNA. Several other housekeeping genes can be used i.e. the Glyceraldehyde-3-Phosphate Dehydrogenase (GAPDH) gene or the Collagen Type II Alpha 1 Chain (COL2A1) gene. Following, methylation status (qualitative measurements) and methylation levels (quantitative measurements) of diabetes-related genes, i.e. INS, IAPP, GCK, KCNJ11 and ABCC8 were analyzed using methylation-dependent SYBR Green-based PCR (MSP) assays. For all genes, primers specific for methylated (m) and unmethylated (u) alleles were either newly designed using the MethPrimer(26) program or were based on bibliography with some modifications. Primer sequences are provided in Table 2. Assay conditions are presented in Table 3.

TABLE 3
Assay conditions

| | Annealing temperature (° C.) |
|---|---|
| Assay | meth/unmeth |
| ACTB | 60 |
| INS | 62/62 |
| IAPP | 62/60 |
| GCK | 65/65 |
| KCNJ11 | 58/60 |
| ABCC8 | 58/58 |

METH methylated,
UNMETH unmethylated

Analytical sensitivity of developed PCR assays was evaluated using serial dilutions of SB-treated methylated and non-methylated DNA controls while efficiency using serial dilutions of the SB-treated DNA controls in H₂O. The analytical sensitivity of all our developed assays was found to be 0.1% in the detection of methylated DNA molecules in a background of unmethylated DNA and 0.5% in the detection of unmethylated DNA molecules in a background of methylated DNA. The efficiency of all our developed assays ranged between 93-96%.

Quantification of methylation can be implemented by different formulas/methods. In one embodiment, methylation in a sample was estimated using the formula:

$$1 - \frac{1}{1 + {2}^{( - Δ Ct )}} \times 100 \% ,$$

where ΔCt=Ct_{unmethylated}−Ct_{methylated}(27). Following, the percentage of methylation was multiplied by the concentration of total serum ccfDNA.

In other embodiments we have used alternative formulas for the quantification of methylation of the selected genes with similar results:
The relative quantity (RQsample) of demethylated alleles using the 2^{-ΔΔCT} method where the ΔΔCT values were generated for each target after normalization by reference gene values(28).
The demethylation index was calculated using the formula: 2^{(methylated Ct-demethylated Ct)} (5).

The acquired data referring to experimental liquid biopsy parameters (including diabetes-related gene methylation ccfDNA parameters) as well and as the lifestyle/clinical/personal/demographic subject data, forming a 2D matrix (i.e. samples/subjects in rows, parameters in columns), were stored in computer memory storage or in cloud to be analyzed. Alternatively, the data can be inserted manually in a computer software.

The results obtained were initially tested using the Kolmogorov-Smirnov test to check for normality in distribution of continuous data. Due to lack of normality in data, non-parametric statistics were used for comparisons between groups. The Spearman correlation coefficient was used as a measurement of correlation for continuous variables. The predictive power of the experimental liquid biopsy parameters was tested using Receiver operating characteristic (ROC) curve analysis and Area Under the Curve (AUC) metric. In all tests performed, statistical significance was set at two-sided p value <0.050. Standard statistical analysis was carried out with the SPSS version 21.0 statistical software package for Windows (IBM—SPSS Inc., USA).

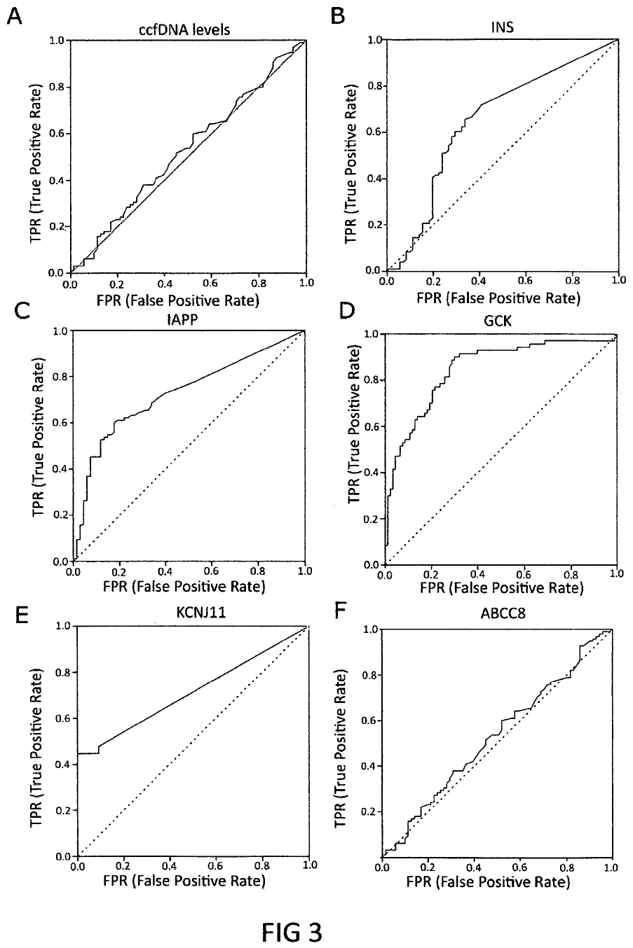

INS, IAPP, GCK and KCNJ11 methylation was significantly different in T2DM patients as compared to the healthy subjects (p=0.001, p<0.001, p<0.001 and p<0.001, respectively) while ABCC8 methylation didn't differ between groups (p=0.534) (FIG. 2). ROC curve analysis showed that GCK methylation could provide very good discrimination between T2DM patients and healthy subjects (AUC 0.848 [95% CI 0.787-0.910]) (FIG. 3D) while LAPP and KCNJ11 methylation could offer good discrimination between groups (AUC 0.727 [95% CI 0.649-0.805] and AUC 0.712 [95% CI 0.619-0.806], respectively) (FIGS. 3C and E). INS methylation showed poor discrimination capacity between patients and controls (AUC 0.650 [95% CI 0.562-0.737]) (FIG. 3B).

Following, data were analyzed by ML tools. Multiple ML algorithms have been employed to build models/classifiers/biosignatures over our dataset to discriminate T2DM from healthy subjects. In one embodiment, our novel liquid biopsy-based data were analyzed by an innovative AutoML tool, namely JADBio version 1.2.8 (https://www.jadbio.com/)(29) in order to produce diagnostic/monitoring models of clinical value. The JADBio tool employed for this analysis performs and compares automatically all standard, best-practices, and advanced ML techniques and produces the optimal best performing model along with the best interpretable one(29). Given a 2D matrix of data, JADBio automatically preprocesses data (Mean Imputation, Mode Imputation, Constant Removal, Standardization), performs feature selection by employing LASSO or Statistical Equivalent Signatures (SES) algorithms, tries several algorithms (i.e. Classification Random Forests, Support Vector Machines (SVM), Ridge Logistic Regression and Classification Decision Trees) and thousands of algorithmic configurations, selects the best performing model and estimates the out-of-sample model's performance after bootstrap correction and cross validation and provides several visualizations(29). For our AutoML classification analysis, we used extensive model tuning effort, we chose the AUC metric for optimization of performance and we set classifier maximum size to five features. The predictive power of the model was assessed using AUC and average Precision (aka area under the Precision-Recall curve) metrics.

In one embodiment, AutoML technology JADBio, was applied to produce diagnostic/monitoring tools/models/biosignatures/classifiers based on the ccfDNA experimental parameters, diabetes-related gene methylation and lifestyle/personal/clinical/demographic subject data.

The AutoML technology JADBio also conducted statistical analysis based on logistic regression model and the results confirmed the models built by the other ML algorithms.

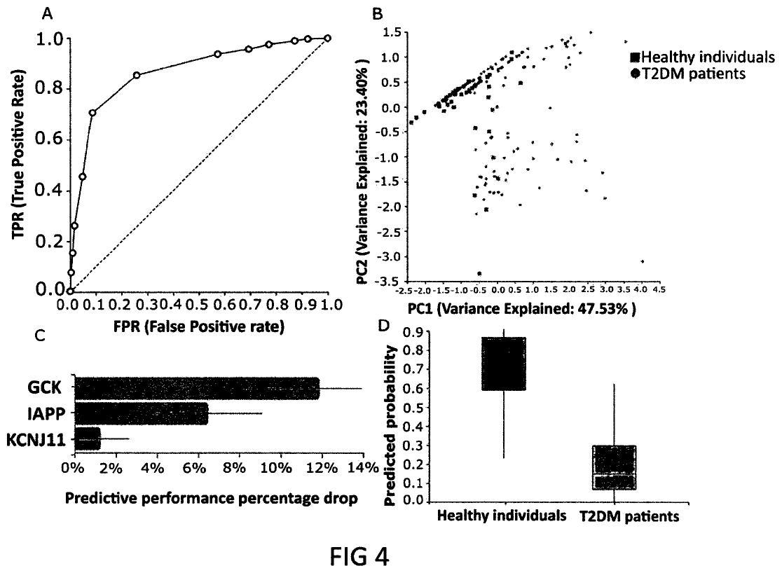

We used AutoML analysis, to predict T2DM versus health from the acquired liquid biopsy parameters (diabetes-related gene methylation and ccfDNA parameters) without and with the lifestyle/personal/clinical/demographic subject data (age, gender, BMI, etc.). In the former case, models build including GCK gene methylation detected in ccfDNA plus at least one other pancreatic gene methylation, demonstrated significantly improved discriminating performance. In specific, the best performing combinations were i) GCK and LAPP, ii) GCK and KCNJ11, iii) GCK, KCNJ11 and IAPP (FIG. 4), showing high performance with AUC ranging from 0.849 (95% CI 0.803-0.892) to 0.892 (95% CI 0.848-0.934).

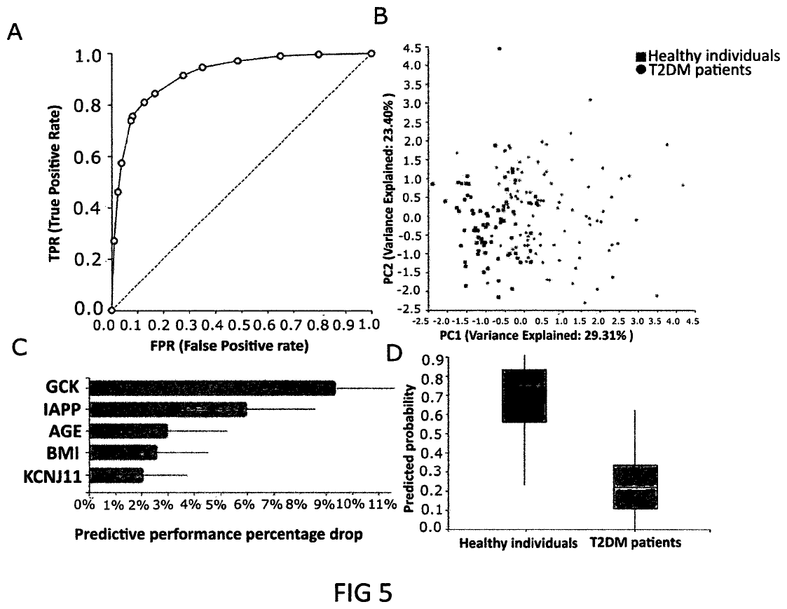

In the latter case, when liquid biopsy parameters (diabetes-related gene methylation and ccfDNA parameters) were combined with subjects' lifestyle/personal/clinical/demographic data, discriminating capacity of AutoML built models was even higher. This AutoML analysis produced a best performing five-feature biosignature via Classification Random Forests algorithm that was able to discriminate extremely well between diabetes patients and healthy subjects with an AUC of 0.927 (95% CI 0.874-0.967) and an average Precision of 0.951 (95% CI 0.914-0.980). Biosignature's features included GCK, LAPP and KCNJ11 methylation as well as age and BMI. Best performing biosignature performance is presented in FIG. 5. A best interpretable five-feature biosignature was also built via Ridge Logistic Regression algorithm reaching an AUC of 0.915 (95% CI 0.868-0.957) and an average Precision of 0.941 (95% CI 0.901-0.975). This biosignature's features included GCK, IAPP and KCNJ11 methylation, smoking status and BMI.

This discriminating performance is higher than any other previously reported or used generally in diabetes pathological entities. For example, LAPP methylation was able to discriminate recent onset T1DM patients and age-matched unrelated healthy subjects with an AUC of 0.866 (95% CI 0.720-1.010)(8). Other studies leveraged demographical, clinical and laboratory data such as age, sex, BMI, glycated hemoglobin (HbAlc), hypertension, smoking, glucose etc. and employed ML tools for predicting diabetes with promising results(19, 20, 30, 31). The predictive ability of the models built however (AUC ranging from 0.826 to 0.872) were inferior to those presented here (AUC reaching 0.927). Only the recent publication of Kaur et al. that analyzed the PIMA Indian Diabetes dataset consisted only of female patients, produced five different predictive models with AUC range from 0.850-0.920(32). The superiority of our model here relies on the use of both sexes in our cohort, and the incorporation of experimental beta-cell specific gene parameters, suitable to detect and monitor beta-cell death.

In addition, it had been shown that GCK methylation can present an early biomarker for β-pancreatic cell destruction(9). Our model incorporates this biomarker with the maximal feature importance in the model's performance, which however is dramatically increased by including also other selected pancreatic gene methylation biomarkers and other parameters. These corroborated findings considered together, support the value of the herein model in the early detection of diabetes onset.

In further embodiments, biomarkers from a subject suspected of having or having said disease are used as input parameters to the trained as previously mentioned prediction models to produce a score indicative for clinical prognosis or diagnosis of Diabetes T2DM. The biomarkers used as input were the same in number and type used to train the predictive model.

The input data in one embodiment are resulting from a method comprising of:
Providing a biological fluid sample from a subject suspected of having or having said disease
Directly quantifying circulating cell free DNA (ccfDNA)
Extracting ccfDNA
Treating the said DNA to convert demethylated cytosines to uracils while sparing the methylated cytosines
Performing a methylation specific detection based on PCR or sequencing-based technology and preferably quantitative methylation specific PCR, to measure the methylation of diabetes-related genes and preferably pancreatic β-cell genes and more preferably of the genes GSK and/or LAPP and/or KCNJ11 and combinations thereof

In further embodiments the data are stored in at least one non-transitory processor-readable storage medium on a computer system or on the cloud and used as input in the predictive model.

### REFERENCES

1. Saeedi P, Petersohn I, Salpea P, Malanda B, Karuranga S, Unwin N, et al. Global and regional diabetes prevalence estimates for 2019 and projections for 2030 and 2045: Results from the International Diabetes Federation Diabetes Atlas, 9<sup>th</sup> edition. Diabetes research and clinical practice. 2019; 157.
2. Kharroubi A T, Darwish H M. Diabetes mellitus: The epidemic of the century. World J Diabetes. 2015; 6(6):850-67.
3. Rahier J, Guiot Y, Goebbels R M, Sempoux C, Henquin J C. Pancreatic beta-cell mass in European subjects with type 2 diabetes. Diabetes, obesity & metabolism. 2008; 10 Suppl 4:32-42.
4. Butler A E, Janson J, Bonner-Weir S, Ritzel R, Rizza R A, Butler P C. Beta-cell deficit and increased beta-cell apoptosis in humans with type 2 diabetes. Diabetes. 2003; 52(1):102-10.
5. Akirav E M, Lebastchi J, Galvan E M, Henegariu O, Akirav M, Ablamunits V, et al. Detection of beta cell death in diabetes using differentially methylated circulating DNA. Proceedings of the National Academy of Sciences of the United States of America. 2011; 108(47):19018-23.
6. Husseiny M I, Kaye A, Zebadua E, Kandeel F, Ferreri K. Tissue-specific methylation of human insulin gene and PCR assay for monitoring beta cell death. PloS one. 2014; 9(4):e94591.
7. Herold K C, Usmani-Brown S, Ghazi T, Lebastchi J, Beam C A, Bellin M D, et al. beta cell death and dysfunction during type 1 diabetes development in at-risk individuals. The Journal of clinical investigation. 2015; 125(3):1163-73.
8. Olsen J A, Kenna L A, Spelios M G, Hessner M J, Akirav E M. Circulating Differentially Methylated Amylin DNA as a Biomarker of beta-Cell Loss in Type 1 Diabetes. PloS one. 2016; 11(4):e0152662.
9. Sklenarova J, Petruzelkova L, Kolouskova S, Lebl J, Sumnik Z, Cinek O. Glucokinase Gene May Be a More Suitable Target Than the Insulin Gene for Detection of beta Cell Death. Endocrinology. 2017; 158(7):2058-65.
10. Lehmann-Werman R, Neiman D, Zemmour H, Moss J, Magenheim J, Vaknin-Dembinsky A, et al. Identification of tissue-specific cell death using methylation patterns of circulating DNA. Proceedings of the National Academy of Sciences of the United States of America. 2016; 113(13):E1826-34.
11. Usmani-Brown S, Lebastchi J, Steck A K, Beam C, Herold K C, Ledizet M. Analysis of beta-cell death in type 1 diabetes by droplet digital PCR. Endocrinology. 2014; 155(9):3694-8.
12. Fisher M M, Perez Chumbiauca C N, Mather K J, Mirmira R G, Tersey S A. Detection of islet beta-cell death in vivo by multiplex PCR analysis of differentially methylated DNA. Endocrinology. 2013; 154(9):3476-81.
13. Gahan P B. Biology of circulating nucleic acids and possible roles in diagnosis and treatment in diabetes and cancer. Infectious disorders drug targets. 2012; 12(5):360-70.
14. Bergman Y, Cedar H. DNA methylation dynamics in health and disease. Nature structural & molecular biology. 2013; 20(3):274-81.
15. Davenport T, Kalakota R. The potential for artificial intelligence in healthcare. Future healthcare journal. 2019; 6(2):94-8.
16. Myszczynska M A, Ojamies P N, Lacoste A M B, Neil D, Saffari A, Mead R, et al. Applications of machine learning to diagnosis and treatment of neurodegenerative diseases. Nature Reviews Neurology. 2020; 16(8):440-56.
17. Schaefer J, Lehne M, Schepers J, Prasser F, Thun S. The use of machine learning in rare diseases: a scoping review. Orphanet Journal of Rare Diseases. 2020; 15(1):145.
18. Kourou K, Exarchos T P, Exarchos K P, Karamouzis M V, Fotiadis D I. Machine learning applications in cancer prognosis and prediction. Computational and Structural Biotechnology Journal. 2015; 13:8-17.
19. Zhang L, Wang Y, Niu M, Wang C, Wang Z. Machine learning for characterizing risk of type 2 diabetes mellitus in a rural Chinese population: the Henan Rural Cohort Study. Scientific Reports. 2020; 10(1):4406.
20. Muhammad L J, Algehyne E A, Usman S S. Predictive Supervised Machine Learning Models for Diabetes Mellitus. SN Computer Science. 2020; 1(5):240.
21. Karaglani M, Gourlia K, Tsamardinos I, Chatzaki E. Accurate Blood-Based Diagnostic Biosignatures for Alzheimer's Disease via Automated Machine Learning. Journal of clinical medicine [Internet]. 2020 2020/09//; 9(9).
22. Markaki M, Tsamardinos I, Langhammer A, Lagani V, Hveem K, Røe OD. A Validated Clinical Risk Prediction Model for Lung Cancer in Smokers of All Ages and Exposure Types: A HUNT Study. EBioMedicine. 2018; 31:36-46.
23. Panagopoulou M, Karaglani M, Balgkouranidou I, Biziota E, Koukaki T, Karamitrousis E, et al. Circulating cell-free DNA in breast cancer: size profiling, levels, and methylation patterns lead to prognostic and predictive classifiers. Oncogene. 2019; 38(18):3387-401.
24. American Diabetes Association. 2. Classification and Diagnosis of Diabetes. Diabetes Care. 2015; 38(Supplement 1):S8.
25. Hattermann K, Mehdorn H M, Mentlein R, Schultka S, Held-Feindt J. A methylation-specific and SYBR-green-based quantitative polymerase chain reaction technique for 06-methylguanine DNA methyltransferase promoter methylation analysis. Analytical biochemistry. 2008; 377(1):62-71.
26. Li L C, Dahiya R. MethPrimer: designing primers for methylation PCRs. Bioinformatics (Oxford, England). 2002; 18(11):1427-31.
27. Lu L, Katsaros D, de la Longrais I A, Sochirca O, Yu H. Hypermethylation of let-7a-3 in epithelial ovarian cancer is associated with low insulin-like growth factor-II expression and favorable prognosis. Cancer research. 2007; 67(21):10117-22.
28. Livak K J, Schmittgen T D. Analysis of relative gene expression data using real-time quantitative PCR and the 2(-Delta Delta C(T)) Method. Methods (San Diego, Calif). 2001; 25(4):402-8.
29. Tsamardinos I, Charonyktakis P, Lakiotaki K, Borboudakis G, Zenklusen J C, Juhl H, et al. Just Add Data: Automated Predictive Modeling and BioSignature Discovery. bioRxiv. 2020:2020.05.04.075747.
30. Farran B, Channanath A M, Behbehani K, Thanaraj T A. Predictive models to assess risk of type 2 diabetes, hypertension and comorbidity: machine-learning algorithms and validation using national health data from Kuwait—a cohort study. 2013; 3(5):e002457.
31. Lai H, Huang H, Keshavjee K, Guergachi A, Gao X. Predictive models for diabetes mellitus using machine learning techniques. BMC Endocrine Disorders. 2019; 19(1):101.
32. Kaur H, Kumari V. Predictive modelling and analytics for diabetes using a machine learning approach. Applied Computing and Informatics. 2020.
33. US20160369340A1, Winthrop University Hospital, 2016-12-22.
34. US20210087630A1, Yissum Research Development Co Of Hebrew University Of Jerusalmem Ltd Hadasit Medical Research Services and Development Co Yissum Research Development Company of Hebrew University of Jerusalem, 2021-3-25
35. US20170308981A1, Blecker Saul; INDEPENDENCE BLUE CROSS; NEW YORK UNIVERSITY; Nigam Somesh; Razavian Narges Sharif; Schmidt Ann Marie; Smith-McLallen Aaron; Sontag David, 2017-10-26
36. Tsamardinos I. et. al. “Just Add Data: Automated Predictive Modeling and BioSignature Discovery, bioRXiv. 5 May 2020, pp. 1-46
37. Sanabilalihassan Ahmed et. al., “The role of DNA methylation in the pathogenesis of type 2 diabetes mellitus”, Clinical Epigenetics Biomed Central LTD, London, UK, vol. 12, no 1, 11 Jul. 2020 pp. 1-23
38 Kaur Harleen et. al., Predictive modelling and analytics for diabetes using a machine learning approach, Applied Computing and Informatics, 28 Jul. 2020
39 Farooq Syed et al., Circulating unmethylated and DNA fragments provide evidence of possible islet cell death in youth with obesity and diabetes, Clinical Epigenetics vol 12, no 1, 31 Jul. 2020, pp. 1-15

1. A computer implemented method for monitoring pancreatic β-cell destruction indicative of prediction, prognosis and diagnosis of type 2 Diabetes Mellitus (T2DM), said method comprising:
training a machine learning tool to minimize false positives and to maximize true positives using input parameters and a training dataset, the machine learning tool running on a computer system comprising a CPU processor and storage memory,
said training dataset including data from patients diagnosed with T2DM and healthy subjects,
using said machine learning tool to create a prediction model that predicts, given at least two or more input parameters, pancreatic beta-cell destruction indicative of the onset or progression of T2DM,
storing the prediction model in at least one non-transitory processor-readable storage medium on a computer system or on the cloud,
and classifying a new case as positive or negative concerning beta-pancreatic cell destruction, based on the said prediction model,
wherein the input parameters comprising at least of two gene methylation biomarkers (biomarker based on the methylation status of a gene) as detected in circulating cell free DNA.

2. A computer implemented method according to claim 1 wherein the gene methylation biomarkers detected in circulating cell free DNA used as input parameters are obtained by a method comprising:
Providing a biological sample that has already been obtained and stored from a subject having said disease and from a subject known not to have the disease (healthy)
Directly quantifying circulating cell free DNA (ccfDNA)
Extracting ccfDNA
Treating the said DNA to convert demethylated cytosines to uracils while sparing the methylated cytosines
Performing a methylation detection method to measure gene methylation.

3. A computer implemented method according to claim 1 or 2, wherein the input parameters comprising of methylation of the genes INS (insulin), IAPP (Islet Amyloid Polypeptide-Amylin), GCK (Glucokinase), KCNJ11 (Potassium Inwardly Rectifying Channel Subfamily J Member 11) and ABCC8 (ATP Binding Cassette Subfamily C Member 8) and their combination and preferably the GSK and/or IAPP and/or KCNJ11 and their combinations.

4. A computer implemented method according to any of the claims 1 to 3 wherein the machine learning tool is an automated machine learning tool.

5. A method according to any to any of the claims 1 to 4, wherein said machine learning tool is using one of the following algorithms: Decision Tree, k-Nearest Neighbors (k-NN), Gradient Boosting Machine (GBM), linear kernel Support Vector Machine (SVM-linear), Radial Basis Function (RBF) kernel Support Vector Machine, Artificial Neural Network (ANN), Multifactor Dimensionality Reduction (MDR), naive Bayes, Classification And Regression Tree (CART) and preferably Support Vector Machine (SVM) or (Classification) Random Forest (RF) or Logistic Regression (LR).

6. A method according to any of the claims 1 to 5, wherein as input parameters are used methylation measurements of the said genes expressed either qualitatively or quantitatively as indexes of methylation levels and preferably calculated by any of the following quantification methods/formulas and combinations thereof:
The relative quantity (RQsample) of demethylated alleles using the 2^{-ΔΔCT} method where the ΔΔCT values were generated for each target after normalization by reference gene values.
The demethylation index was calculated using the formula: 2^{(methylated Ct-unmethylated Ct)}.
The percentage of methylation in a sample using the formula:
$$1 - \frac{1}{1 + {2}^{( - Δ Ct )}} \times 100 \% ,$$
where ΔCt=Ct_{unmethylated}−Ct_{methylated}

7. A method according to claim 6, wherein the reference gene is any housekeeping gene and preferably ACTB or GADPH or COL2A1 gene.

8. A method according to any of the previous claims 1-7, wherein the biological sample comprises a body fluid and preferably blood and more preferably serum or plasma.

9. A method according to any of the claims 2 to 8, wherein the methylation specific detection to measure the methylation and methylation levels of the said genes is conducted by sequencing or by PCR-based technology.

10. A method according to any of the previous claims 1-9, wherein the input parameters further comprising lifestyle and/or personal and/or demographic and/or clinical and/or clinicopathological data of the subjects and more preferably the BMI and/or the smoking status and/or the age of the subjects and combinations thereof.
